(19) United States
(12) Patent Application Publication
(10) Pub. No.: US 2005/0054747 A1
(43) Pub. Date: Mar. 10, 2005

(54) Long-lasting scented paint and method for making same

(76) Inventors: Oscar D. Sandlin, Indianapolis, IN (US); Bill Oltman, Indianapolis, IN (US)

Correspondence Address: Bryan S. Redding, Suite 800, 8888 Keystone Crossing Blvd., Indianapolis, IN, 46240, US

(21) Appl. No.: 10/656,553

(22) Filed: Sep. 6, 2003

(51) Int. Cl. C09D 11/00; A61L 2/00

(52) U.S. Cl. 523/102; 106/31.02

(57) ABSTRACT

The present invention discloses a long-lasting scented paint mixture and method for creating the scented paint, whereby a scenting agent is introduced and mixed into a standard paint composition whereby the resultant mixture will produce an odor or scent that will last after the paint has been applied and has been allowed to fully cure.

### CROSS-REFERENCE TO RELATED APPLICATIONS

Not Applicable

### STATEMENT REGARDING FEDERAL SPONSORED RESEARCH OR DEVELOPMENT

Not Applicable

### REFERENCE TO SEQUENCE LISTING, ETC

Not Applicable

### BACKGROUND

The present invention relates to paints with long-lasting scents and the method for creating them. More particularly, the present invention relates generally to long-lasting scented paints, and the method for producing them, whereby a particularly chosen scent lasts long after the application and drying of the paint.

In our lives, paints are used in any number of ways including for protective, decorative and communicative purposes (i.e. signs or signals). Generally, basic paint is made by creating a mixture of a binder, sometimes a resin or polymer, plus a solvent, to dissolve or suspend the binder and to give the paint the correct consistency, one or more pigments, to give color to the paint, and a vehicle which acts to hold the pigment to the painted surface. Additionally, additives can also be included to provide certain desirable characteristics, such as, quicker drying, harder surface or to change the PH of the paint. Paints can be of a variety of types, among other things, oil-based, water-based, enamels, etc. Due to the nature of the solvent and/or other chemicals used in its production, paint generally has a noxious and offensive odor which is particularly strong during and immediately after application. Background art discloses the use of masking agents and scents to mask the odor of fresh paint, that is while it is being applied, and which scents are meant to last only until the paint fully dries or cures. While the present invention does include a scenting agent, it does not, and is not meant to, mask the odor of the paint prior to curing. Further, related art has disclosed the use of scented inks in various packaging, artistic and novelty applications, but not as a paint for a large surface.

The present invention relates to scents and odors, which are obviously directed to the sense of smell. The sense of smell is one of the five human senses and arguably one of the most important. While research and information about the sense of smell is continuing, it is readily apparent from the proliferation of scent based businesses (candles, oils, lotions, incenses, powders, creams, etc.) that the sense of smell is a central one to our lives. In fact, scents set our moods, evoke memories, assists with the sense of taste and can even have healing properties. This premise is a cornerstone of the practice of aromatheraphy, where it is believed certain scents can provide relief from headaches, muscle and neck pain, arthritis, insomnia, bronchitis and others. Scent can also be for protection, as seen when it is used in insect repellants.

It is for the above reasons that people at home, and in their businesses, often seek to introduce scents and odors by various means. Normally, this requires some external mechanism such as an air freshener, perfume, oils, candle, incense, potpourri or other method to impart the desired scent into the room. In addition to the costs associated with these mechanisms, they sometimes also involve an element of danger or risk, as is seen in the use of candles (open flame), incenses (unattended burning), potpourri pots (risk of fire from drying up and overheating). Thus, it would be preferable if consumers and business owners could impart a long-lasting scent of their choosing through the use of something that will be in most rooms anyway, that is, paint.

Therefore, a problem not addressed by the relevant background art, is that people may desire the introduction of a long-lasting scent of their choosing into a room, without the costs or dangers attached to the use of external devices or mechanisms. For the foregoing reasons, there is a need for a long-lasting scented paint and for a method of making the same.

### BRIEF SUMMARY

The present invention is directed to the needs and desires noted above for a long-lasting scented paint having features of wide selection of scents, at a low cost, and with ease of production, by being able to introduce the scenting agent at any step in the well known process of making paint, including after the paint has been applied but has not yet dried. Furthermore, it is an object of this invention that the introduction of the scenting agent also allows for other additives, that provide desirable characteristics, still being able to be added to paint and to maintain the desirable characteristics.

It is a further object of this invention that a user be allowed to adjust the amount of scent desired, prior to the application, and also that the scents can also be of utility, that is, in the instance where the scenting agent is one having insect repellant qualities, such as citronella.

It is a further object of this invention that the scenting agent can be selected such that a change in the scent or strength could be seen based upon characteristics of the room or the surface upon which the paint is applied, that is, perhaps dependent upon temperature or humidity.

It is a further object of this invention that the scent can be chosen to provide information about the room or surface to those individuals that not be able to see the room or surface, such as blind or visually impaired persons. This would aid them in moving about structures, which may otherwise provide unnecessary obstacles to their movement.

These and other features, aspects and advantages of the present invention will become better understood with reference to the following description, appended claims and accompanying drawing.

### BRIEF DESCRIPTION OF THE DRAWINGS

FIG. 1: shows a schematic of a claimed embodiment whereby the scenting agent is added to the finish paint.

FIG. 2: shows a schematic of a claimed embodiment whereby the scenting agent is added to the solvent that is then mixed with the paint to create finish paint.

FIG. 3: shows a schematic of a claimed embodiment whereby the scenting agent is added to the paint following manufacture.

FIG. 4: shows a schematic of a claimed embodiment whereby the scenting agent is added to the pigment during the manufacturing process.

FIG. 5: shows a schematic of a claimed embodiment whereby the scenting agent is added to the binder during the manufacturing process.

FIG. 6: shows a schematic of a claimed embodiment whereby the scenting agent is added to the solvent used during the manufacturing process.

FIG. 7: shows a schematic of a claimed embodiment whereby the scenting agent is added to the vehicle during the manufacturing process.

FIG. 8: shows a schematic of a claimed embodiment whereby the scenting agent is added to one or more additives during the manufacturing process.

### DESCRIPTION

The long-lasting scented paint of this invention may be produced by combining a selected pigment, binder, solvent and a vehicle, and in some cases additives, with a selected scenting agent. The selected pigment is any suitable for use in paint, which are known by those skilled in the art. Likewise, the binder is either a resin or polymer that is suitable for the making of paint, which will also be well known by those skilled in the art. Additionally, the solvent can be any number of things, including, water, oil, or various liquids, as long as it suitable to be in paint, of which, will be known by those skilled in the art. Further, a suitable vehicle should be selected. The vehicle can be any number of chemicals but should have the property of holding the pigment to the painted surface until and after curing of the paint. These are commonly known to those skilled in the art. Lastly, in some cases, additives can also be included, and these include any number of chemicals, which provide for desirous characteristics such as quick-drying, additional hardness of the resulting surface, and others. These chemicals are widely known by those skilled in this area.

As to the scenting agent, this can be any number of scent extracts or oils which could be natural or man-made in any number of forms, that is a solid or liquid form. The scent used will be at the discretion of the end-user but the form may be limited by the nature of the production of the scent.

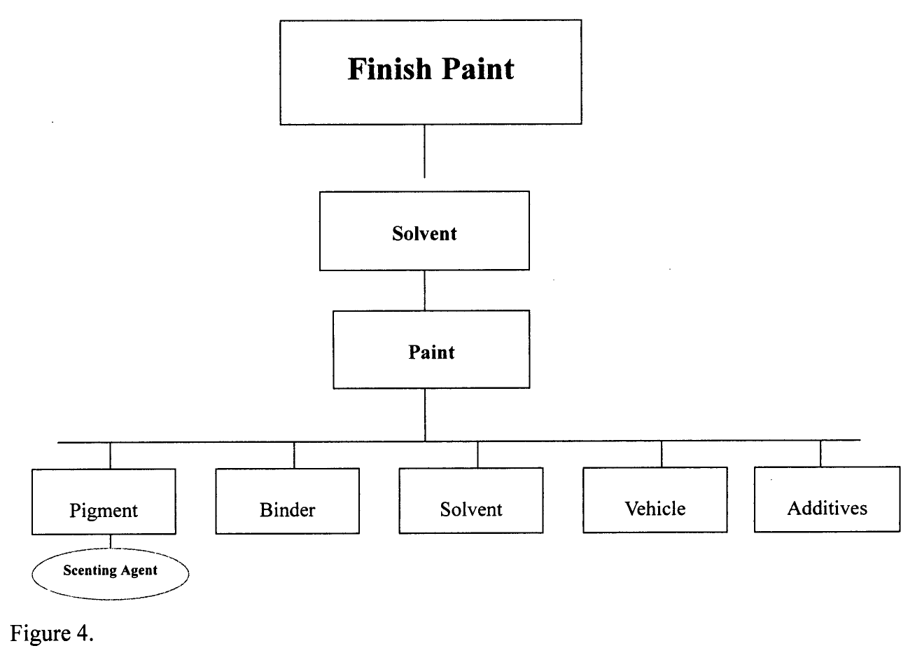

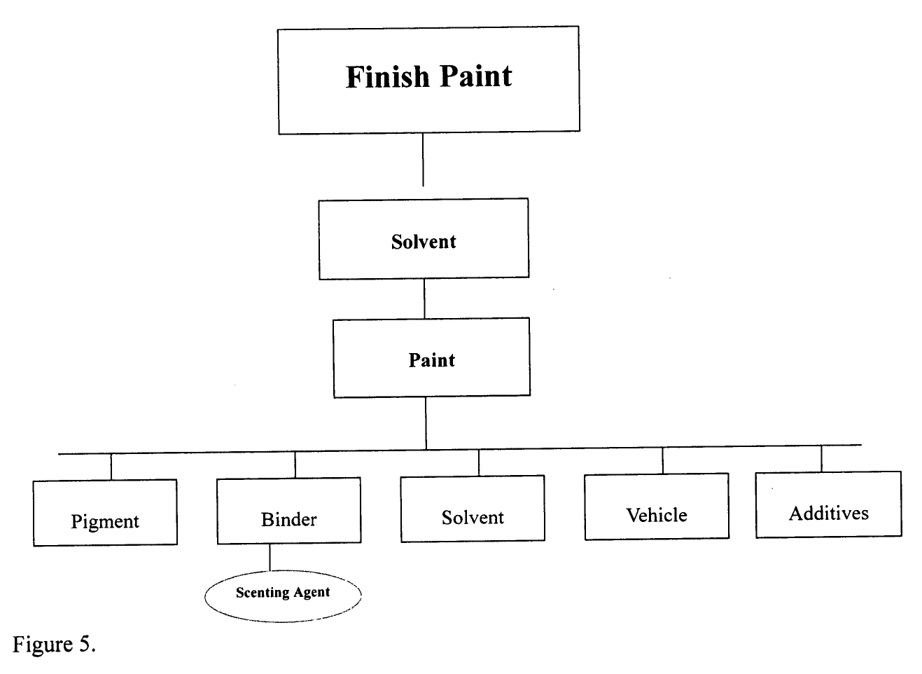

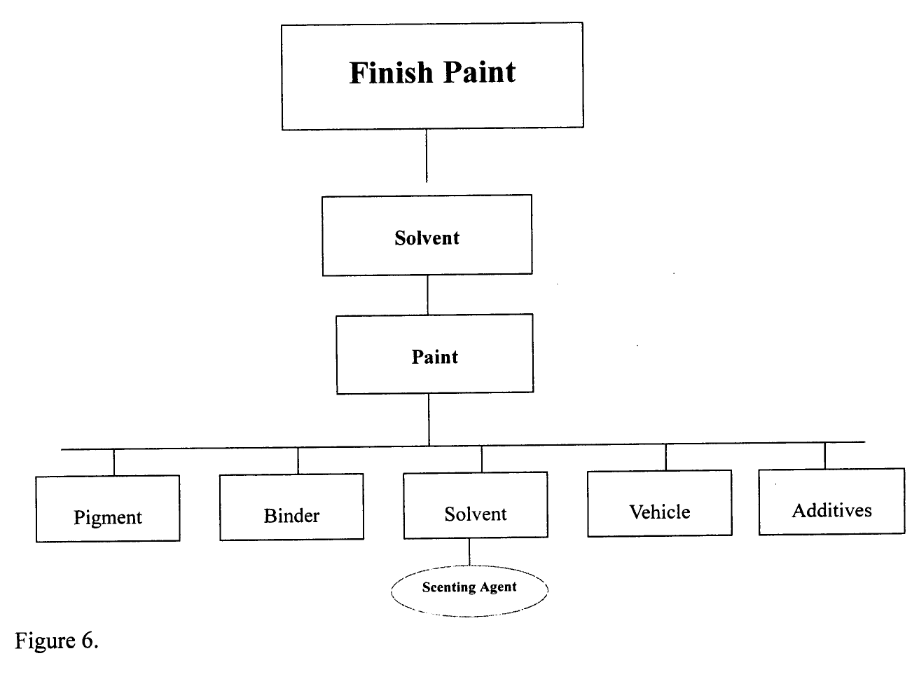

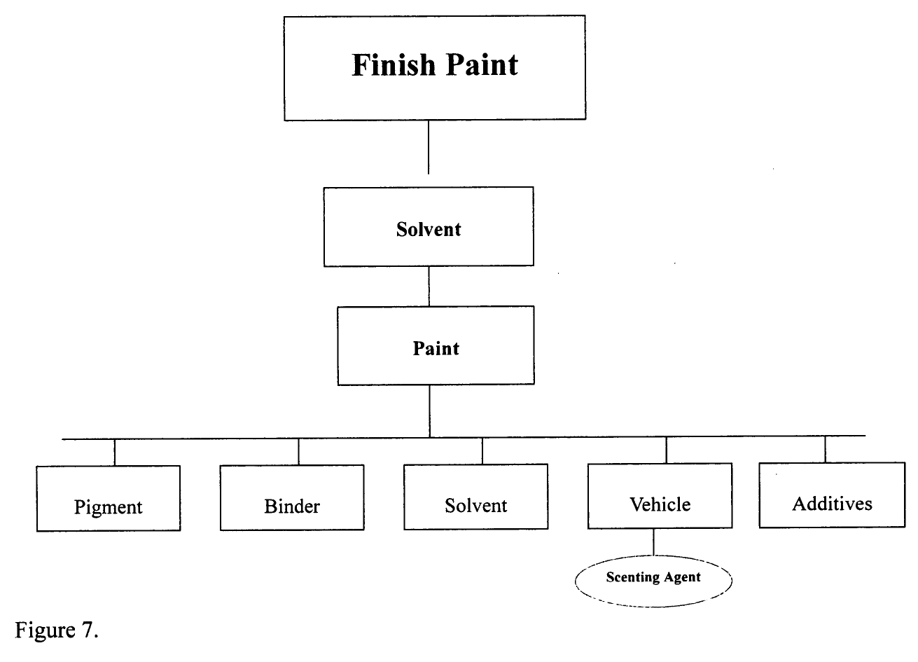

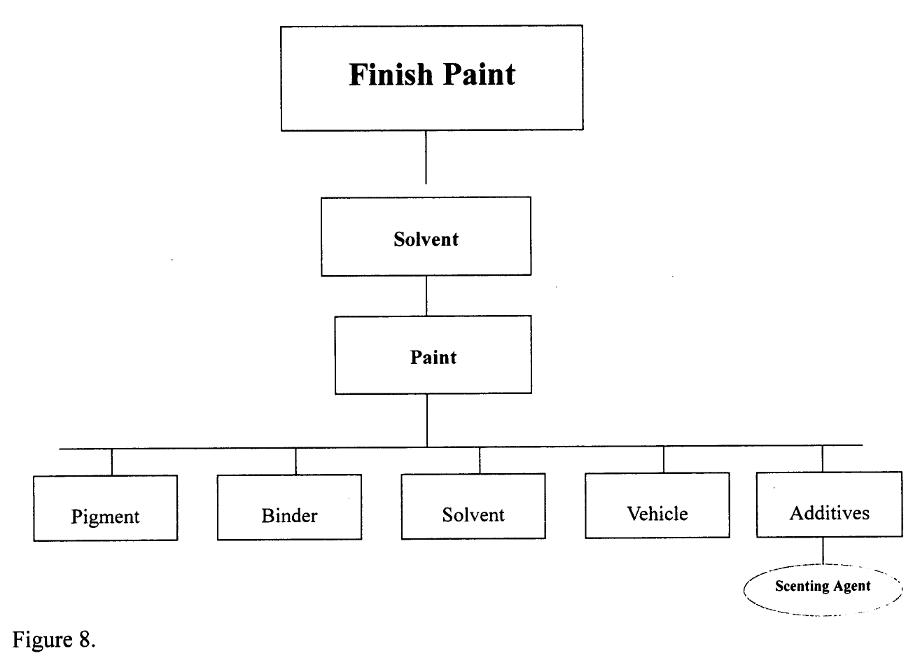

Initially, a pigment is selected, which is prepared in a conventional manner for introduction into a paint mixture. In one embodiment of the invention, it is possible, that the scenting agent could be added to the pigment, and the two combined, prior to the binder and solvent being added, however this is only one possible way in which the scenting agent can be introduced into the paint. This is demonstrated in FIG. 4. In additional embodiments, the scenting agent is added to the one of the other elements of the paint during the manufacturing process. A few of these additional embodiments are shown by FIGS. 5-8. In whichever embodiment is selected, the raw ingredients, including the scenting agent, is then mixed in a conventional manner, such as a mixing vat or drum, known to those skilled in the art.

The amount of the scenting agent is variable depending upon the strength of the scent sought, but should not be of such a volume to cause a detrimental effect upon the inherent paint characteristics necessary to meet the requirements sought for said paint. Notwithstanding, the scenting agent must be of sufficient strength and volume to remain as a scent after the paint has been applied and allowed to fully cure.

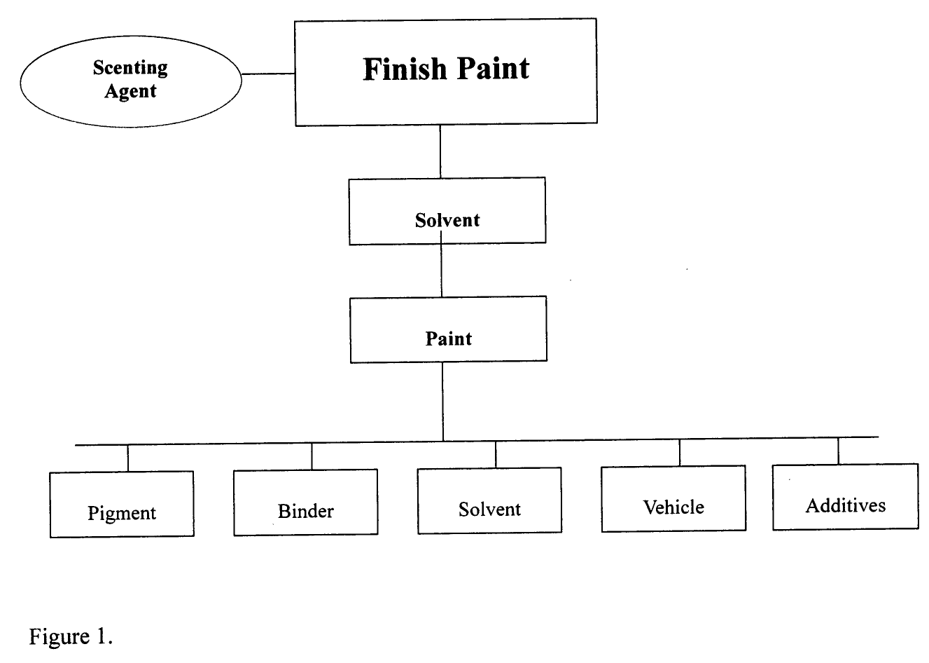

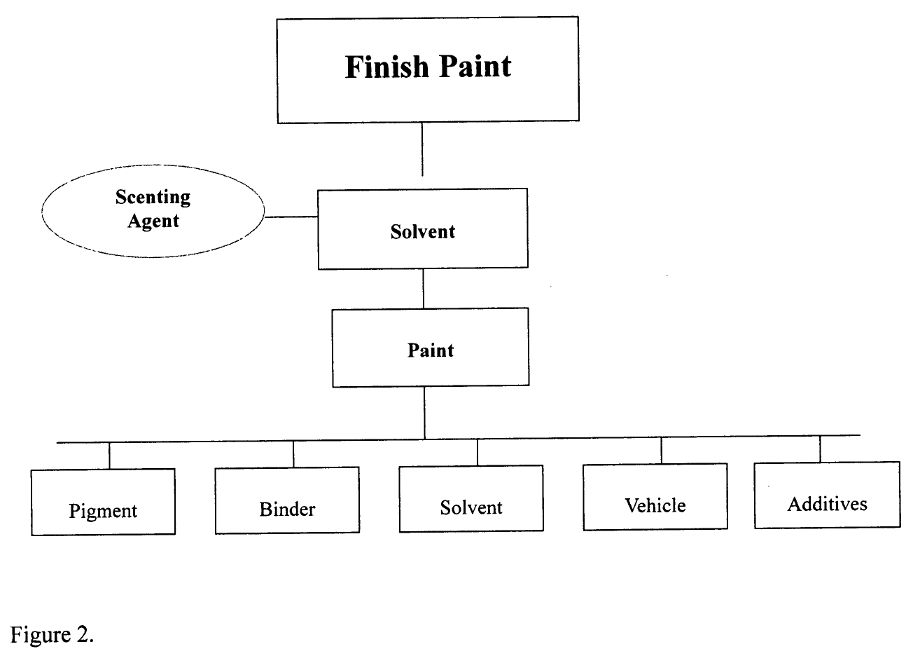

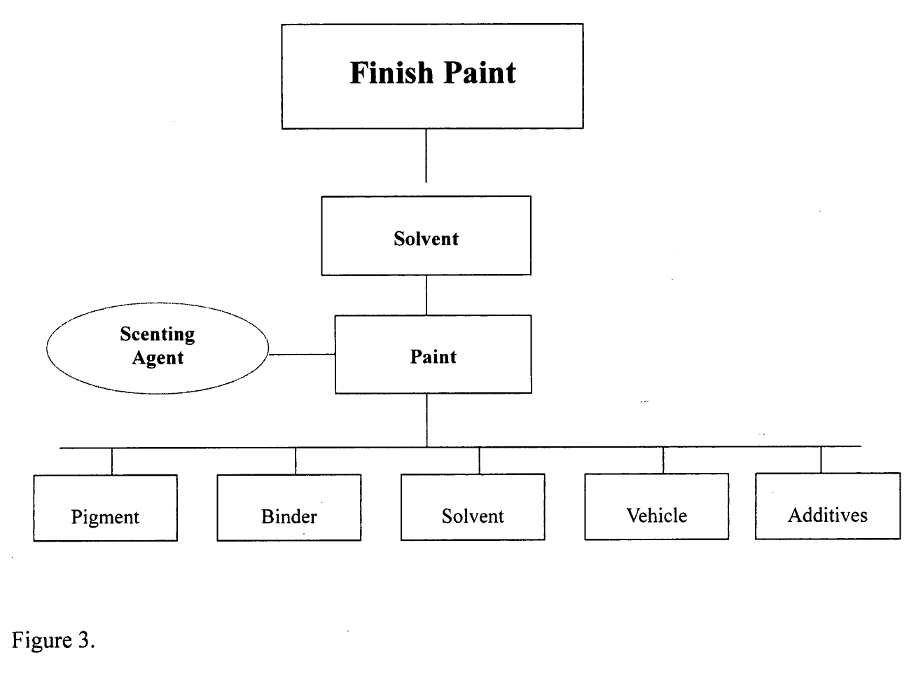

In the preferred embodiment, the scenting agent is added after the paint has been manufactured and is ready to apply. Normally, after paint is manufactured it is then distributed for resale to consumers at which time additional solvent is normally added and, in some cases, a final chosen color or pigment, is introduced to the paint. As shown in FIG. 1, the scenting agent may be introduced to the finished paint at the time it is prepared for use, or even by the end-user or consumer, by manual introduction of the scenting agent. In another embodiment, the scenting agent could be added to the solvent, prior to mixing, which produces the finish paint, as shown in FIG. 2. In another embodiment, the scenting agent could be added to the paint, following manufacture, but before the introduction of the solvent used to produce finish paint, as demonstrated by FIG. 3.

In any of the above embodiments, the scenting agent would be added to the elements noted, in an amount to provide the desired scent level, and the mixture would then be mixed or agitated in a sufficient manner to ensure thorough distribution of the scenting agent throughout the selected element.

It is also possible to introduce a scenting agent which could have additional beneficial characteristics, such as being repellant to insects. This would enable the painting of an area around a patio that would provide the users of the patio an area free from insects or other pests. Likewise, it would be possible to include a scenting agent that could impart information to those being present in the room or surface painted with the long-lasting scented paint. For example, this could be used to assist those with visual impairments to establish their location within a facility or to allow movement within a facility.

Furthermore, a scenting agent could be selected and introduced based upon the fact that the odor or scent associated with the scenting agent, changes depending upon the ambient temperature or humidity of the room or surface painted with the long-lasting scented paint.

In another preferred embodiment, a long-lasting paint “scent line” could be produced, which provides a set of basic paint colors with standard scents, in varying degrees. These could also be produced on a custom-order basis, based upon specific customer requests.

Although the present invention has been described in considerable detail with reference to certain preferred versions thereof, other versions are possible. Therefore, the spirit and scope of the appended claims should not be limited to the description of the preferred versions contained herein.

1. A method of making a long-lasting scented paint mixture comprising:
a) selecting a pigment suitable for use in paint;
b) selecting a binder suitable for use in paint;
c) selecting a suitable solvent;
d) selecting a suitable vehicle;
e) selecting a scenting agent of characteristics such that it will provide continued scent and odor after the paint is applied to a selected surface and allowed to dry;
f) mixing or agitating the pigment, binder, solvent, vehicle and scenting agent until thorough dispersion all ingredients takes place.

2. The method of claim 1 whereby the pigment and selected scenting agent are mixed or agitated prior to being introduced and mixed with the other elements.

3. The method of claim 1 whereby the binder and selected scenting agent are mixed or agitated prior to being introduced and mixed with the other elements.

4. The method of claim 1 whereby the solvent and selected scenting agent are mixed or agitated prior to being introduced and mixed with the other elements.

5. The method of claim 1 whereby the selected scenting agent is mixed or agitated into the previously prepared paint mixture consisting of the elements noted.

6. The method of claim 1 whereby the selected scenting agent is mixed or agitated with one or more additives, selected to provide certain desired characteristics, prior to being introduced and mixed with the other elements.

7. A method of making a long-lasting scented finish paint comprising:
a) selecting a paint;
b) selecting a suitable solvent;
c) selecting a scenting agent of characteristics such that it will provide continued scent and odor after the paint is applied to a selected surface and allowed to dry;
d) mixing or agitating the paint, solvent and scenting agent until thorough dispersion of all ingredients takes place.

8. The method of claim 7 whereby the solvent and selected scenting agent are mixed or agitated prior to being introduced and mixed with the other elements.

9. The method of claim 7 whereby the paint and selected scenting agent are mixed or agitated prior to being introduced and mixed with the other elements.

10. The method of claim 7 whereby the solvent and paint have been combined to create a finish paint and then the selected scenting agent is mixed or agitated into the finish paint.

11. The method of claim 7 whereby the selected scenting agent and an additional pigment are mixed or agitated prior to being introduced to the other elements.

12. A method of making a long-lasting scented paint comprising:
a) selecting an unscented paint;
b) applying the unscented paint to a selected surface;
c) selecting a scenting agent;
d) prior to the time the unscented paint has dried on the selected surface, introduce the selected scenting agent to the surface whereby it will adhere to the wet paint and remain on the selected surface along with the paint.

13. A scented paint mixture made according to the process of claim 1.

14. A scented paint mixture made according to the process of claim 7.

15. A scented paint mixture made according to the process of claim 12.

16. The mixture of claim 1 in which said scenting agent includes cinnamon.

17. The mixture of claim 7 in which said scenting agent includes cinnamon.

18. The mixture of claim 12 in which said scenting agent includes cinnamon.

19. The mixture of claim 1 in which said scenting agent is one having insect repellant qualities, such as, citronella.

20. The mixture of claim 7 in which said scenting agent is one having insect repellant qualities, such as, citronella.

21. The mixture of claim 12 in which said scenting agent is one having insect repellant qualities, such as, citronella.

22. The mixture of claim 1 whereby the scenting agent is selected for purposes of imparting information about the room or surface upon which the mixture is applied to those not able to visually see and/or examine the room or surface.

23. The mixture of claim 7 whereby the scenting agent is selected for purposes of imparting information about the room or surface upon which the mixture is applied to those not able to visually see and/or examine the room or surface.

24. The mixture of claim 12 whereby the scenting agent is selected for purposes of imparting information about the room or surface upon which the mixture is applied to those not able to visually see and/or examine the room or surface.
